(19) United States
(12) Patent Application Publication
(10) Pub. No.: US 2010/0191074 A1
(43) Pub. Date: Jul. 29, 2010

(54) DISTRIBUTED MULTI-CHANNEL PHYSIOLOGICAL MONITORING AND ANALYZING SYSTEM

(76) Inventor: Chang-An Chou, Taipei (TW)

Correspondence Address: Chang-An Chou, 3F, No. 100, Sec. 3. Mingsheng E. Rd., Taipei, 105, TW

(21) Appl. No.: 12/676,910

(22) PCT Filed: Sep. 12, 2008
(86) PCT No.: PCT/CN2008/001611

§ 371 Date: Mar. 8, 2010

(30) Foreign Application Priority Data
Sep. 13, 2007 (CN) 200710154744.9
Jun. 6, 2008 (CN) 200810108325.6

(51) Int. Cl. A61B 5/00

(52) U.S. Cl. 600/301

(57) ABSTRACT

A distributed multi-channel physiological monitoring and analyzing system including an assembly of plural biosignal collecting units and a processing unit is provided. The plural biosignal collecting units are implemented to acquire multiple physiological signals from an user. Each biosignal collecting unit includes at least a sensor/electrode for acquiring at least one channel of physiological signal, a processor for data processing, a wireless module for data transmission and communication, a memory for storing the acquired physiological signals, and a real-time clock module for providing a time base to sample the physiological signal and providing time axis information of the physiological signal. Moreover, the processing unit includes a wireless module for receiving physiological signals from the biosignal collecting units for on-line monitoring. Furthermore, a time matching operation is performed by aligning the time axis information among the processing unit and plural biosignal collecting units, and one single set of time-correlated physiological data is formed based on the acquired multiple physiological signals and the result of the time matching operation for further analysis.

### FIELD OF THE INVENTION

The present invention is related to a distributed multi-channel physiological monitoring and analyzing system, and more particularly to a distributed multi-channel physiological monitoring and analyzing system which can provide higher mobility and comfortability to the user, as well as an improved remote control capability.

### BACKGROUND OF THE INVENTION

Nowadays, people seem to have developed higher standards and demands with respect to the improved life quality. Therefore, in addition to basic physiological monitoring, operation convenience also becomes more important to the user and the manufacturer, especially for the more complicated system like multi-channel physiological monitoring and analyzing system, for example, there are many multi-channel physiological diagnostic systems used in sleep researches have focused on convenience.

Usually, the conventional diagnostic system used in sleep study, known as polysomnographic (PSG) system, restricts the patient's motion seriously since there are numerous wires connected between the sensors/electrodes attached on the patient and the main unit aside, and this situation may significantly reduce the patient's motivation for accepting the examination.

Afterward, the ambulatory multi-channel physiological diagnostic system has been developed for sleep study, in which the main unit is carried by the patient for shortening the connecting wires of the electrodes/sensors. However, since on-line monitoring is important in physiological diagnostics, especially for PSG system, normally, the main unit still has to connect to a separate monitoring device. Therefore, the moving range of the patient is limited thereby.

Furthermore, the wireless technology is also adapted to the ambulatory diagnostic system, so as to separate the system into two parts: wireless wearable part and non-wearable part. The wearable part is connected to sensors/electrodes for retrieving physiological signals from the body and transmitting thereof. The non wearable part is a wireless receiver for receiving physiological signals from the wearable part for being further processed. Some examples are: Crystal Monitor 20-B, Crystal Monitor 20-S, Sapphire PSG and Sleep Scout manufactured by CleveMed (Cleveland Medical Devices Inc.), AURA® PSG Wireless/Ambulatory Systems manufactured by GRASS technology, Somté PSG manufactured by Compumedics, WEE-100 AirEEG manufactured by NIHON KOHDEN, SOMNOscreen™ and SOMNOscreen™ EEG 10-20 manufactured by SOMNOmedics, etc.

As an example, Crystal Monitor 20-B consists of one patient unit and one computer unit. The patient unit is to be carried by the user and connected to the electrodes/sensors and the computer unit is for connecting to the computer, and further, the patient unit and the computer unit can wirelessly communicate with each other. Therefore, through the wireless communication therebetween, the physiological signals acquired by the patient unit can be wirelessly transmitted to the computer via the computer unit, so as to further achieve the physiological monitoring during the sleep study.

AURA® PSG Wireless/Ambulatory Systems and Somté PSG do the same way. They both use wireless communication between patient unit and computer unit for enabling a user to move anywhere in the receiving range thereof.

However, although the wireless communication eliminates the wire connecting between the patient unit and the computer unit and provides the patient a great mobility, the numerous, entangled and easily be pulled connecting wires for electrodes/sensors still remain on the patient's body surface. And, the similar scenario is also encountered in other physiological monitoring systems as adopting the wireless transmission technology. Thereby, the problem of wiring complexity and the loading it brings to the user still exists.

For all these reasons, how to make the multi-channel physiological diagnostic system more comfortable, user-friendly and applicable so as to satisfy the future demands thereof has become an important issue.

The object of the present invention is to provide a distributed multi-channel physiological monitoring and analyzing system. By using distributed architecture, equipped memory and innovative synchronization process, this system solves most technical problems in the present stage, such as, sensors and electrodes must be connected to the same main unit, and the wiring for connection is too complicated, etc.

Another object of the present invention is to provide a distributed multi-channel physiological monitoring and analyzing system which can minimize the extra load added to the user's body for providing a more comfortable wearing experience and also can simplify the procedure of installation compared to the conventional multi-channel physiological diagnostic system, thereby making an adaptable long-term physiological monitoring system for homecare possible.

Further another object of the present invention is to provide a distributed multi-channel physiological monitoring and analyzing system which can not only record the physiological signals but also wirelessly send thereof out for on-line monitoring, so that as integrating with the network, a remote control for physiological monitoring becomes practical.

### SUMMARY OF THE INVENTION

For achieving the objects described above, the present invention provides a distributed multi-channel physiological monitoring and analyzing system including an assembly of plural biosignal collecting units and a processing unit. The plural biosignal collecting units are implemented to acquire multiple physiological signals from an user. Each biosignal collecting unit includes at least a sensor/electrode for acquiring at least one channel of physiological signal, a processor for data processing, a wireless module for data transmission and communication, a memory for storing the acquired physiological signals, and a real-time clock module for providing a time base to sample the physiological signal and providing time axis information of the physiological signal. Moreover, the processing unit includes a wireless module for receiving physiological signals from the biosignal collecting units for on-line monitoring. Furthermore, a time matching operation is performed by aligning the time axis information among the processing unit and plural biosignal collecting units, and one single set of time-correlated physiological data is formed based on the acquired multiple physiological signals and the result of the time matching operation for further analysis.

The real time clock module is characteristic of maintaining accurate and identical time intervals, so that in the present invention, the time base provided by the real time clock module for sampling the physiological signal can remain stable during the whole signal acquisition, and particularly, since the time base of each biosignal collecting unit is identical, the sampling intervals of physiological signals can remain the same.

Additionally, the real time clock module also can generate time axis information of physiological signal. By the accuracy of time base, it can obtain the time axis information, for example, the start time and/or stop time, the time duration, and the time stamps on specific events, etc. Therefore, a time alignment among the processing unit and the biosignal collecting units can be performed for achieving the time matching operation.

Hence, through performing the time matching operation, the distributed design of the present invention can be achieved with the accuracy of one single set of time correlated physiological data being maintained.

Besides, the distributed architecture according to the present invention not only can reduce wiring complexity, but also can offer the user a more comfortable wearing experience and a greater mobility during signal acquisition.

Preferably, the memory is a removable memory and the result of the time matching operation is stored therein, so that when the processing unit receives the physiological signals from the removable memories, the result of the time matching operation is also received thereby.

Preferably, the time matching operation is executed before, during and/or after physiological signal acquisition.

According to the present invention, the processing unit can have different configurations, for example, the processing unit can be integrated with one of the plural biosignal collecting units, can be implemented as a computer, such as a personal computer and a notebook, with the wireless module thereof being built therein or externally connected thereto, can be composed of a computer and a embedded device which communicate with each other, or can be implemented to be a standalone device for receiving and processing the physiological signals.

Advantageously, the system of the present invention can further include an adapter device intermediate between the processing unit and the biosignal collecting units, and an event button for being pressed to mark special events, such as, certain physiological conditions or unexpected situations. Moreover, the system can be implemented to have an alarm function based on the physiological signals acquired by the biosignal collecting units. In addition, the system also can be connected to a network for achieving remote operations.

### BRIEF DESCRIPTION OF THE DRAWINGS

A more detailed understanding of the invention may be had from the following description of a preferred embodiment, given by way of example, and to be understood in conjunction with the accompanying drawings, wherein:

FIG. 1 is a schematic view showing a distributed multi-channel physiological monitoring and analyzing system according to the present invention;

FIG. 2 is a block diagram of a biosignal collecting unit in a preferred embodiment according to the present invention;

FIG. 3 is a flow chart showing the operation procedure according to a preferred embodiment of the present invention;

FIG. 4 is a schematic view showing a first example of the present invention;

FIG. 5 is a schematic view showing a second example of the present invention;

FIG. 6 is a schematic view showing a third example of the present invention;

FIG. 7A is a schematic view showing a preferred embodiment of the present invention with one adapter device;

FIG. 7B is a schematic view showing a preferred embodiment of the present invention with plural adapter devices; and

FIG. 8 is a schematic view showing the system of the present invention connecting to a remote system via a network.

### DETAILED DESCRIPTION OF THE PREFERRED EMBODIMENTS

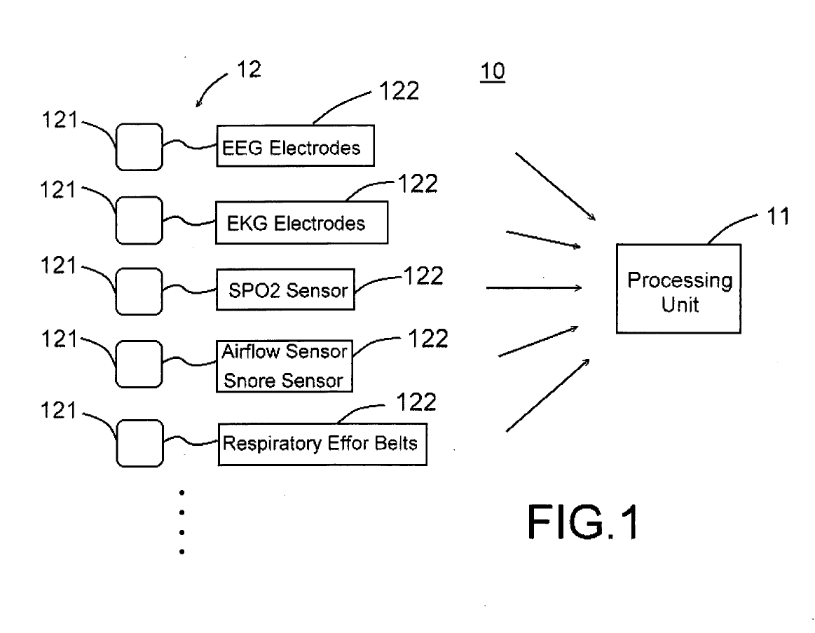

The present invention provides a distributed physiological monitoring and analyzing system 10 with multiple channels, as shown in FIG. 1, including an assembly 12 of biosignal collecting units 121 and a processing unit 11, wherein the biosignal collecting units 121 are installed on an user for acquiring physiological signals, and the processing unit 11 is used to receive the physiological signals from the biosignal collecting units 121 and to integrate them into one single set of time-correlated physiological data.

According to the present invention, each biosignal collecting unit 121 is connected with at least an electrode/sensor 122 for acquiring at least one channel of physiological signals. Here, the electrode/sensor can be more than one, for example, as shown in FIG. 1, one biosignal collecting unit can be connected with multiple electrodes, multiple sensors, or electrode and sensor for monitoring single or multiple kinds of physiological signals. For example, the biosignal collecting unit can be connected with multiple electrodes for processing multiple lead ECG monitoring, or simultaneously connected with respiratory sensor and snore sensor for monitoring two kinds of physiological signals, or connected with one single finger SPO2 sensor, so that the measured physiological signals can be classified into different groups according to demand difference, such as, according to different body portions, different monitoring purposes, or different types of physiological signals, however, there is no limitation.

Different from the traditional way in which all the sensors/electrodes are connected to one single device (no matter how far the distance is), the purpose of the present invention is to make the execution of biosignal collecting unit independent of other biosignal collecting units, so that each of the plural biosignal collecting units in system according to the present invention can independently gather the physiological signals, and the multi-channel physiological monitoring is achieved by cooperation of all biosignal collecting units. Besides, it is even better that each biosignal collecting unit can simply initiate the biosignal acquisition just after the installation without waiting for other biosignal collecting units' installation, and after all the biosignal collecting units' installations are finished, the initiation of the whole system is automatically completed.

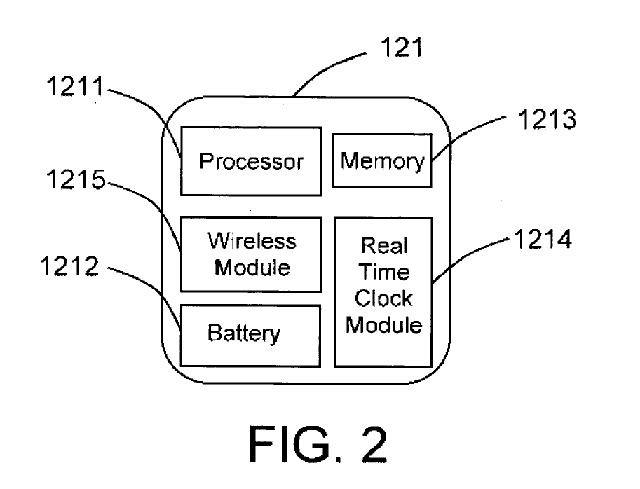

For achieving the purpose described above, in the present invention, the conventional centralized apparatus (no matter placed aside or worn on the patient) which connects to all sensors/electrodes is distributed into plural biosignal collecting units. As shown in FIG. 2, each biosignal collecting unit 121 includes a processor 1211, a battery 1212 for providing electricity without power cord, and a memory 1213 for storing physiological signals. Here, for reducing redundant connecting wires, the wire extended from the biosignal collecting unit is employed to connect with electrode(s)/sensor(s), and there is no wire connection between biosignal collecting units. Therefore, based on the independency, the connecting wires to the electrode/sensor will be located around the biosignal collecting unit, and the conventional long connecting wire is omitted, such as, the finger SPO2 measurement always employs a long connecting wire from the finger across the arm to the apparatus on the chest, or the leg movement detection also employs a connecting wire which goes from leg to heart position. Accordingly, the biosignal collecting unit can be installed at a better position, which not only can lower down the wire interference but also can improve measurement accuracy since longer wires may introduce more noise from the air.

Furthermore, since on-line monitoring is necessary for some physiological monitoring, especially for EEG and PSG monitoring, each biosignal collecting unit 121 of the present invention also can include a wireless module 1215, for providing wireless communication capability, and correspondingly, the processing unit can include a wireless module. Through the wireless module 1215, plural biosignal collecting units 121 can wirelessly and instantly transmit the acquired physiological signals to the processing unit 11 for signal trace display. Besides, the wireless transmission capability also can be employed to achieve a testing procedure, such as, the impedance check for electrode installation.

For example, when carrying out a PSG monitoring, sleep technician may stand aside for monitoring the patient. First, as installing electrodes/sensors, sleep technician will ask the patient to do some actions, such as, to wink his/her eyes, to breathe deeply or to move his/her legs/hands, so that the technician can check the function and also the installation accuracy of the electrodes/sensors. Then, the patient falls asleep, and the technician will observe all the monitored physiological signals of the patient in the recording duration to see if there any unusual situation happens, such as electrode/sensor falls off, so as to help data analysis after recording.

Take EEG monitoring as another example. Usually, an impedance check will be performed after the electrodes are installed. Then, the medical personnel can simply observe the physiological signals, can ask the patient to do some actions or thinking, or can provide some stimulation to the patient, such as, in the situation of biofeedback, for observing the corresponding signal response.

Accordingly, owing to the wireless transmission technology provided in the present invention, the processing unit and the biosignal collecting units can have an instant communication capability, so it is easily to conform to the on-line monitoring requirement.

Besides, because there is the possibility of data lost during wireless transmission, the equipped memory in the biosignal collecting unit 121 is implemented to record the acquired physiological signals no matter the wireless transmission is applicable or not, so that in addition to instantly transmitting acquired data, it also can be implemented to transmit primary or partial data, or transmit data at intervals, or transmit data owing to medical personnel's trigger, which means it can have different transmission modes depending on various conditions, for example, it can be chose to be the trigger mode for saving power since the biosignal collecting units are powered by battery.

Then, at the end of biosignal acquisition, for further analyzing the recorded physiological signals, the processing unit has to access the memories. Here, the processing unit can respectively connect to every biosignal collecting unit for obtaining the recorded signals, or the memory can be configured as removable memory, for example, memory card, so the processing unit can access the data directly through a memory access interface, such as a card reader.

Now, it is important that how these individually acquired signals are synchronized and integrated into one single set of time-correlated physiological data, just like those obtained in the conventional multi-channel physiological diagnostic systems employing one centralized main unit/patient unit.

That's because for most multi-channel physiological monitoring, except the on-line monitoring, a post interpretation and analysis for figuring out the physiological condition is also a major purpose. As in PSG analysis, generally, when the technician needs to mark events, such as, apnea, hypopnea, body turnover, or teeth grinding, he/she has to co-reference to multiple physiological signals for confirming the accuracy. For example, if it has to classify the sleep apnea into central or obstructive type, in addition to observing the breath of the patient, the technician also has to pay attention to other physiological conditions, such as, snore, respiratory effort and oxygen saturation in order to make a correct decision.

Therefore, the present invention provides a time matching operation method for solving this problem, and the key for achieving the time matching operation is the real time clock module 1214 provided in each biosignal collecting unit, as described above. Here, the real time clock module 1214 can be implemented as separate module or built in the processor 1211.

By using the real time clock module, an accurate and stable time base can be provided for sampling the physiological signal in each biosignal collecting unit, and further, in the present invention, the time base of each physiological signal is identical, so that the sampling intervals of all biosignal collecting units can be the same. In addition, the real time clock module is also used to generate the time axis information of physiological signal, and the time matching operation is achieved by aligning the time axis information among the processing unit and the biosignal collecting units.

Therefore, according to the present invention, the real time clock module not only ensures the accuracy of time base, but also defines the time axis information of physiological signals, for example, the start and stop times.

Followings are some examples for explaining the time matching operation, but as known by one skilled in the art, these are only for illustration and not for limitation.

First of all, before further explaining, it should be notified that this time matching operation can be performed in a wireless or wired manner, for example, it can be achieved by the processing unit respectively contacts with plural biosignal collecting units or with the memories thereof, or by the processing unit directly and wirelessly communicates with plural biosignal collecting units. Furthermore, the timing for executing the time matching operation is also unlimited, for example, it can be executed before, during and/or after the biosignal acquisition and even can be performed as the processing unit receives the physiological signals.

In one example, the time matching operation is performed to be direct time synchronization between the time axes of the processing unit and the real time clock modules. That is, the real time clock module in each biosignal collecting unit is adjusted to be identical to the time of the processing unit, and simultaneously, a time stamp will be generated on the physiological signal. Therefore, since the physiological signal is sampled based on the accurate time base provided by the real time clock module, the processing unit can achieve the time matching operation simply by aligning the time stamps.

In another example, the time matching operation is achieved by obtaining a time difference between the time axes of the processing unit and the real time clock module. Here, the time difference can be recorded in the processing unit, and/or the biosignal collecting units, so after receiving the physiological signals, the processing unit can achieve the time matching operation simply by adjusting the time axis of each physiological signal. For example, if the time difference of one physiological signal is −10 minutes, it only has to shift the time axis forward 10 minutes, and if the time difference of one physiological signal is +5 minutes, it only has to shift the time axis backward 5 minutes.

In still another example, the time matching operation is achieved by the processing directly drives the processors of the biosignal collecting units to respectively generate time stamps on the physiological signals, so that the processing unit can execute the time matching operation by aligning the time stamps after receiving the physiological signals.

Of course, different kinds of time matching operations can be used together for further ensuring the accuracy of time relationship of recorded signals.

Moreover, since the real time clock module, other than providing accurate time, is characteristic of extremely power-saving and equipped with independent power source (such as battery or golden capacitor), when the signal acquisition is interrupted, the power is used up, or the battery is replaced during a certain period, the time axis can remain unchanged, for example, the time relationship between the produced time stamp and the real time clock module, or the processed time synchronization can maintain the same. Therefore, when the processing unit receives the physiological signals, even the biosignal collecting units run out of power, the time accuracy still can be remained so as to promise correct physiological signal integration.

Particularly, if the biosignal collecting units complete the time matching operation and the information (physiological signals, time axis information and setting information) is stored in the memories, when the memories are implemented as removable, the processing unit can obtain physiological signals, time and setting information at the same time through one single downloading step from the memory. Especially, it is popular now in using removable memory card and the corresponding card reader is also quite common, so the accessing process can be simplified. Most importantly, the operation becomes more convenient. For example, the patient can visit the doctor only by carrying the memories, and the doctor can realize the result through accessing the memories, and further, if the doctor needs to alter settings and/or parameters of signal acquisition, then he/she can change the settings and/or parameters just through modifying the memories.

Furthermore, in addition to the time matching operation can be performed in different ways, the pattern of the time matching operation among the processing unit and the biosignal collecting units also can have different choices.

For example, the processing unit can simultaneously communicate with all the biosignal collecting units, or the processing unit can communicate with each of the plural biosignal collecting units sequentially, or the processing unit can communicate with one of the biosignal collecting units and the time-matched biosignal collecting unit then communicates with other biosignal collecting units, or the processing unit can communicate with one of the biosignal collecting units and the time-matched biosignal collecting unit communicates with the next biosignal collecting unit until the time matching operations of all biosignal collecting units are completed. Of course, there is no limitation. For instance, the biosignal collecting units can perform the time matching operation with each other first, and then, with the processing unit.

Therefore, the multi-channel physiological monitoring system of the present invention not only provides the primary on-line monitoring, but also achieves post analysis and interpretation through the feature of the time matching operation which ensures the accuracy of synchronization and integration of multiple physiological signals.

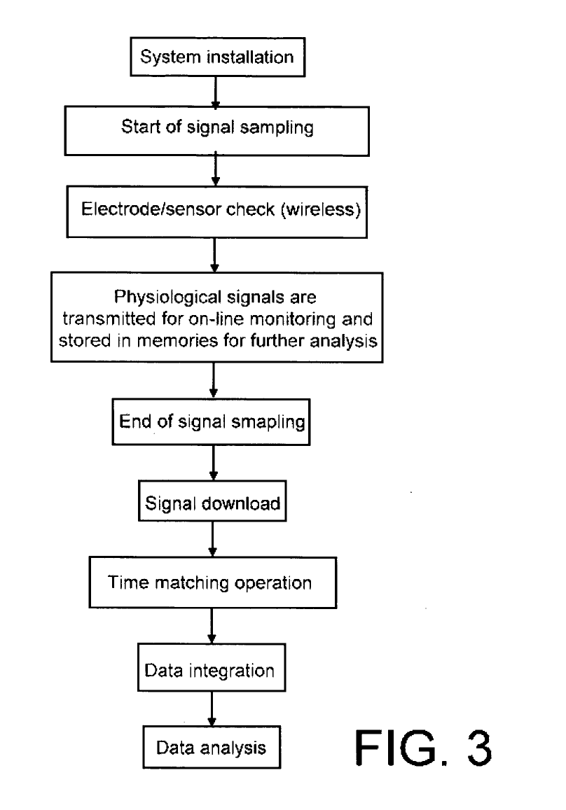

Now, please refer to FIG. 3, which is a flow chart showing the operation procedure according to a preferred embodiment of the present invention. As shown, first, the user installs plural biosignal collecting units on examining locations, for example, the SPO2 sensor is positioned on the finger tip with the biosignal collecting unit positioned on the wrist, or the airflow sensor is disposed between the nose and the mouth with the biosignal collecting unit positioned on the cheek or around the head, wherein particularly, the airflow sensor can share the biosignal collecting unit with the snore sensor, so that two sensors can be initiated together, and also, the number of biosignal collecting units can be reduced. According to the present invention, it can be each biosignal collecting unit is initiated by the power switch thereon, or all the biosignal collecting units are initiated by one thereof or by the processing unit, or all the biosignal collecting units are initiated automatically through setting schedule, or other methods, which are not limited.

Then, a check of the installation accuracy through a wireless communication between the biosignal collecting units and the processing unit, such as impedance check, may be performed.

During biosignal acquisition, the physiological signals acquired by every biosignal collecting unit will be stored in the memory thereof, and most importantly, each physiological signal is sampled based on the time base provided by the real time clock module, which is also used to generate the time axis information thereof. Besides, other than data recording, the acquired physiological signals also will be wirelessly transmitted to the processing unit through the wireless module in each biosignal collecting unit for achieving an on-line monitoring. Here, there is no limitation about the transmission, for example, the contents and frequency thereof both can be decided by the user through the processing unit.

After biosignal acquiring, the biosignal collecting unit with the sensor/electrode can be removed from the user's body, and then, the physiological signals stored in the memories can proceed to the next analysis. Here, the methods for downloading the data in the memories can be achieved by wireless transmission or contact transmission, or even by removable memories.

Then, as receiving the physiological signals, the time matching operation by aligning the time axis information among the processing unit and the biosignal collecting units is performed, so that one single set of time-correlated physiological data can be formed based on the multiple physiological signals from plural individual biosignal collecting units and the result of the time matching operation as finishing the download.

It should be noticed that although in the above description, the time matching operation and the data receiving are packaged in one step (for minimizing the amount of steps), the time for executing the time matching operation is still not limited, as mentioned above, it can be performed before, during and/or after the biosignal acquisition.

Followings are some applicable examples.

### Example 1

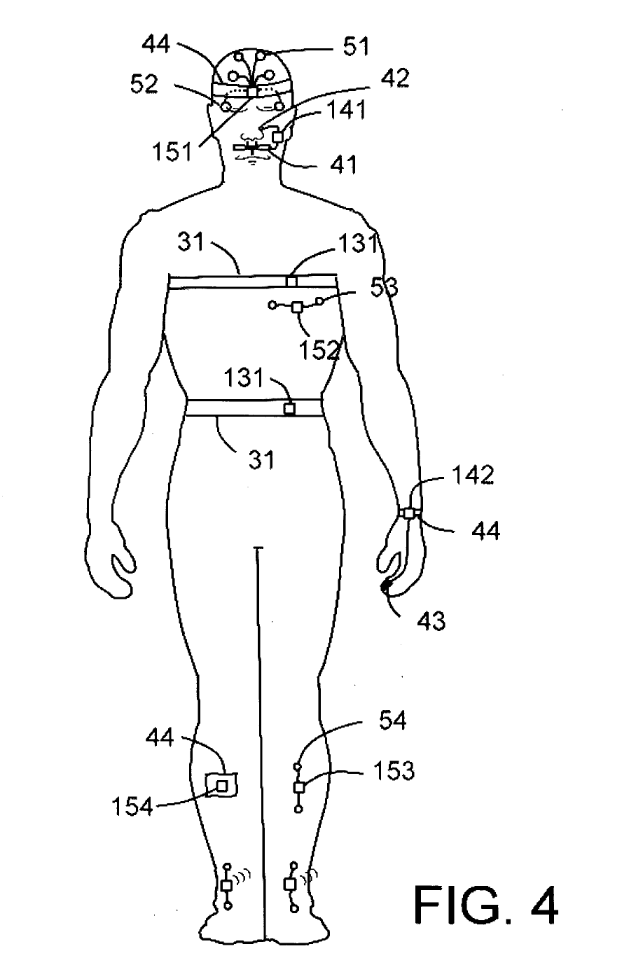

In this example, the multi-channel physiological monitoring and analyzing system according to the present invention is implemented to be polysomnographic (PSG) diagnostic system, which manifests most advantages. As shown in FIG. 4, a biosignal collecting unit 151 with EEG electrodes 51 and EOG electrodes 52 are located on the forehead, a biosignal collecting unit 141 with airflow sensor 41 and snore sensor 42 is located on the cheek, a biosignal collecting unit 152 with ECG electrodes 53 is located on the chest or disposed on one respiratory belt 31, and a biosignal collecting unit 142 with SPO2 sensor 43 is bound at the wrist or adhered to the back of the hand. Further, if it is necessary to measure limb movement, biosignal collecting units 153, 154 with EMG electrodes 54 or position sensor (not shown, usually located in the unit) can be positioned on the leg.

Through the arrangement above, the wiring complexity is minimized, and the moving limitation is greatly reduced. Besides, compared with the conventional centralized diagnostic system, this distributed design not only can cut down the long connecting wire for providing convenience, but also can significantly increase the wearing comfortability for the user. Therefore, owing to the convenience and comfortability, the present PSG system becomes more suitable for home testing purpose than conventional PSG systems, that is, it becomes possible for the user to perform the PSG monitoring at home, on his/her own bed, which is most familiar therewith, and thus, not merely the time traveling to the sleep lab can be save, the result also might be more accurate.

Since the time matching operation can be performed at any time and is based on the accurate time base and the time axis information from the real time clock module, the only operation the user needs to do is to install the biosignal collecting units with electrode/sensor on the body (and press the start button, which might not be needed depending on different applications). Even, the installation can be made as visiting the doctor, and the patient only has to press the start button for initiation after going home.

Then, one-single set of time-correlated physiological data, just like the common PSG data obtained in the conventional PSG system, can be formed based on the received the physiological signals and the result of the time matching operation just after the processing unit finishes receiving physiological signals. So, the patient can just bring the memories to see the doctor, and the doctor can simply get the PSG data through integrating the physiological signals in all memories by the processing unit.

In addition to the operation convenience, the distributed design also promises the independency of each biosignal collecting unit, so that the biosignal collecting unit can be omitted without influencing on other units' operation. Therefore, the user may decide the physiological signals he/she needs to collect. That is, just through the present invention, it will be free to gather different combinations of physiological signal included in this system for conforming to various demands.

### Example 2

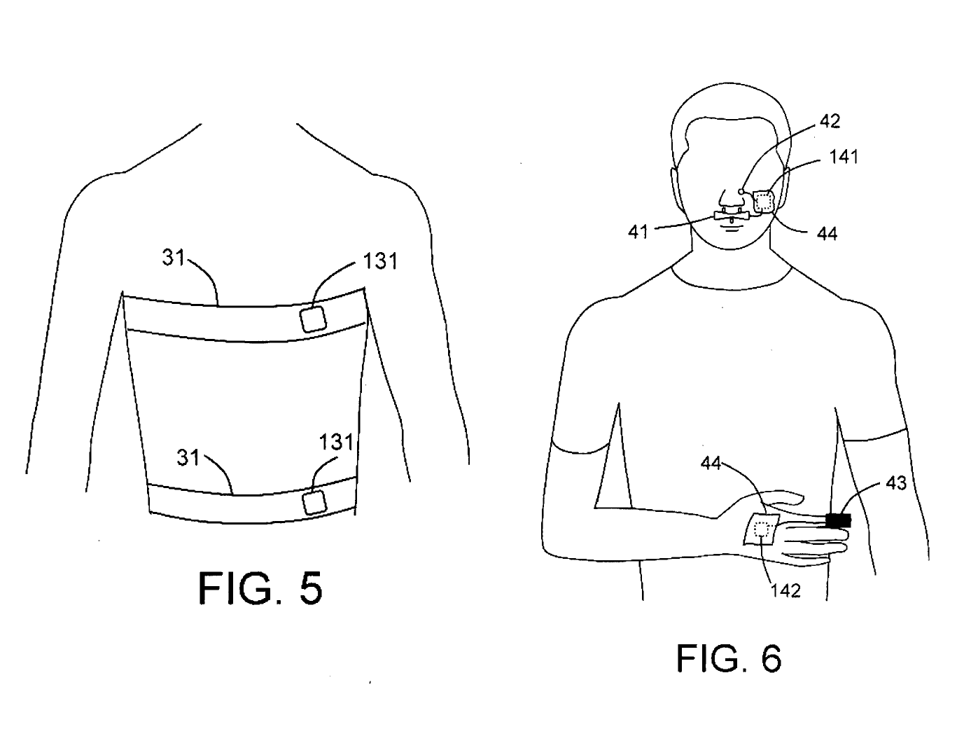

When the present invention is implemented to monitor respiratory effort through two belts 31, it can be one biosignal collecting unit 131 connected to two belts. Alternatively, as shown in FIG. 5, it also can be one belt 131 combined with one biosignal collecting unit 131 for further reducing the connecting wire.

### Example 3

If a screening test of respiration is needed, as shown in FIG. 6, the user can select respiration-related biosignal collecting units, such as, airflow, snore, and respiratory effort, to combine as a screener. As shown, the airflow sensor 41 and the snore sensor 42 are together connected to the biosignal collecting unit 141 owing to the position correlation, the thoracic belt and the abdomen belt are respectively combined with a biosignal collecting unit, and if the oxygen saturation measurement is needed, another biosignal collecting unit 142 with the SPO2 sensor 43 may be mounted on the hand.

After biosignal acquisition, the processing unit can receive the signals in turn or together and then integrate thereof into one single set of time-correlated physiological data. That is, no matter how many physiological signals are received, the integration and synchronization thereof are not influenced only if the time matching operation is executed before, during and/or after the biosignal acquisition.

Accordingly, the present invention carries out a cost-saving architecture. The user can decide the biosignal collecting unit combination, which might be different every time depending on the measuring demands, by himself/herself without being limited by the conventional hardware configuration. Alternatively, at the first time, the user also can just buy the biosignal collecting units for collecting the needed physiological signals, and when other measuring demands present, the user additionally buys other units for combining them together without further setting and difficulty.

Here, the biosignals acquired by the distributed multi-channel physiological monitoring and analyzing system according to the present invention include, but not limited, EEG, EOG, M.S, EMG, A.T. EMG, EKG, respiratory airflow, respiratory effort, SPO2, snore, body position, blood pressure, and body temperature.

Moreover, in one preferred embodiment, the processing unit according to the present invention can be implemented as a general purpose computer, such as personal computer and notebook, with corresponding software installed therein, and the wireless module can be built in the computer or externally connected to the computer, such as a wireless RF dongle. In another preferred embodiment, the processing unit also can be composed of a computer and a embedded device which will communicate with each other for further providing the user the operation convenience. In another further preferred embodiment, the processing unit can be a standalone device with functions of receiving physiological signals from multiple biosignal collecting units and processing thereof. However, these are only examples and there is no limitation for implementing the processing unit, for example, the processing unit also can be implemented to combine with one of the biosignal collecting units. Therefore, there is no limitation to the arrangement, configuration and/or architecture of the present invention, and those embodiments achieving the identical function of the present invention all falls in the scope disclosed by the accomplished claims.

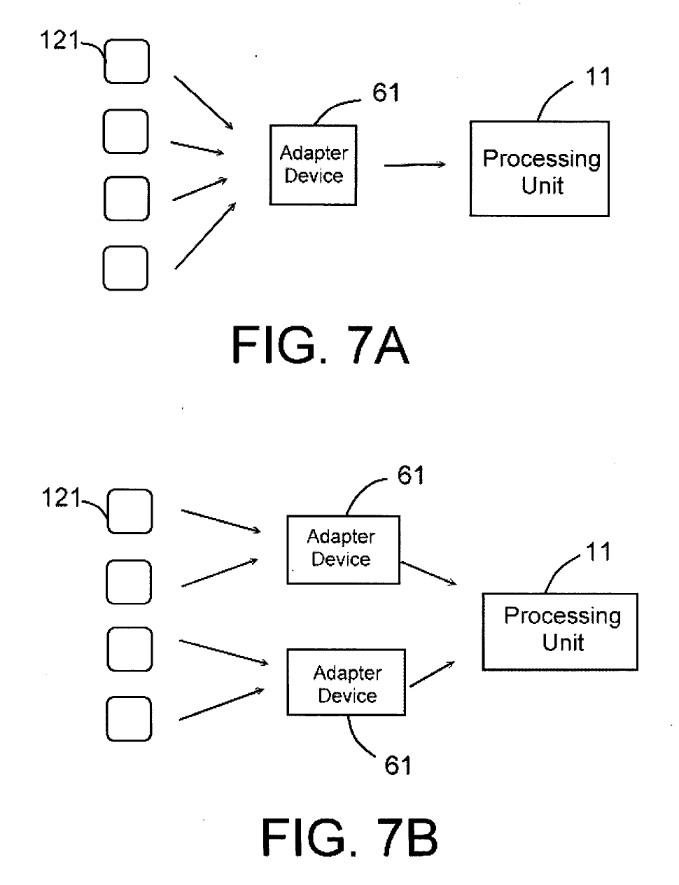

Furthermore, for increasing operation convenience, it also can be, for example, as shown in FIG. 7A, further including at least an adapter device 61, for receiving the physiological signals and then transmitting to the processing unit 11. Further, the adapter device 61 even can replace the processing unit to perform the time matching operation. Besides, of course, it can be implemented to have plural adapter devices 61, as shown in FIG. 7B. Each adapter device 61 can respectively receive part of physiological signals to perform the time matching operation and then transmit the matched physiological data to the processing unit 11, and after receiving the physiological data from plural adapter devices 61, the processing unit 11 performs further time matching operation for forming one single set of time-correlated physiological data.

Moreover, in another embodiment, the system of the present invention also can include an event button (not shown) for being mounted on or put aside the body, or being integrated with one of the biosignal collecting units. When the user feels not comfort, the certain physiological conditions happen or some unexpected situations occur, such as the sensor/unit falls off, the user can mark this event by pressing the event button. Therefore, as analyzing the physiological data, the medical personnel or technician can pay more attention to the marked event. This embodiment is especially beneficial to people who have heart problem and try to find out the problem through long-term monitoring.

In still another embodiment, a button or a device for triggering the time matching operation and/or the system initiation also can be included so as to increase convenience.

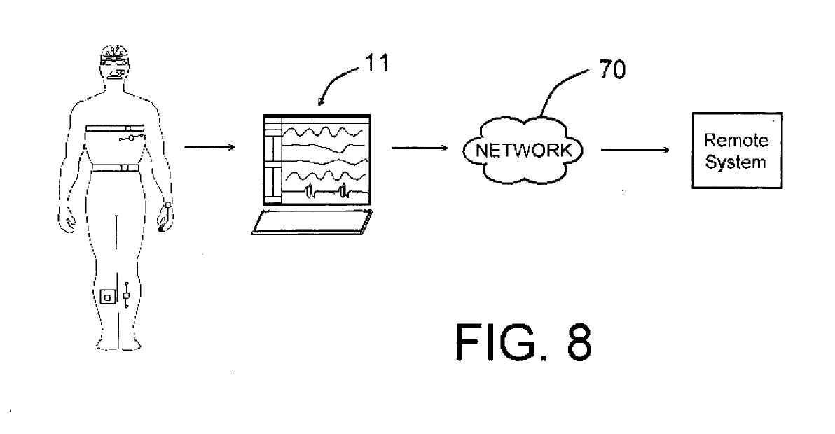

In another aspect of the present invention, the system can be connected with a network, as shown in FIG. 8, for achieving a remote on-line monitoring. For example, through the network, the processing unit can be connected with a remote monitoring system in the hospital or the related service organization, such as, physical examination center, so that the wirelessly received physiological signals by the processing unit can be transmitted to the remote monitoring system. Thus, the medical personnel, such as doctor, nurse, or sleep technician, can directly and instantly realize if the electrode/sensor installation is correct, for example, and inform the user through an audio or video manner. Accordingly, even though the physiological monitoring is performed at home, the accuracy also can be maintained. And, since the remote monitoring is applicable, not only the monitoring labor of the technician can be saved, the user also can save the time for traveling to the hospital or the like.

Through the cooperation between the wireless modules and the network, the system of the present invention can achieve a remote alarm function. For example, an alarm button can be mounted on the biosignal collecting unit for helping the user to ask for help as something emergent happens. In this situation, after the alarm button is pressed, the processor drives the wireless module to transmit an alarm signal to the processing unit, and then, the processing unit sends the alarm signal for informing a remote rescue system. This is particularly advantageous to people who live alone or are physical disabilities. Of course, the alarm button can be formed to be a separate unit.

Alternatively, the alarm signal also can be automatically produced by the system. For example, the processing unit can periodically check the physiological signals transmitted from the biosignal collecting units, which are related to life sign monitoring, such as, units for measuring breath, body temperature, and EKG (heart rate) so that as the physiological signal, e.g., heart rate, is lower or higher than a preset threshold, the processing unit can automatically generate an alarm signal, for example, a sound, for warning people around the user, or the alarm signal can further be transmitted through the network for notifying a remote monitoring personnel or rescue system. This can avoid the situation that the user is incapable of pressing the alarm button or is alone. Besides, for preventing from false-alarm, such as the sensor/electrode falls off, a confirmation mechanism can be employed, e.g., an audio/video communication or a further comparison with other physiological signals, which is not limited.

In the aforesaid, the distributed multi-channel physiological monitoring and analyzing system according to the present invention utilizes distributed plural biosignal collecting units to perform physiological signal acquisitions by storing thereof in memories in the biosignal collecting units, and a processing unit to receive and integrate the acquired physiological signals. Particularly, each biosignal collecting unit includes a real time clock module for providing accurate time base for sampling physiological signals and generating time axis information of physiological signal, and more particularly, the time base of each physiological signal is identical. Moreover, a time matching operation by aligning the time axis information among the processing unit and the biosignal collecting units is provided before, during and/or after the biosignal acquisitions. Therefore, one single set of time correlated physiological data can be built by multiple physiological signals and the result of time matching operation. Accordingly, the distributed plural biosignal collecting units not only cuts down the long wires for reducing the introduced noise thereby and simplifies the wiring complexity, but also provides the user a great moving convenience during biosignal acquisition, and further, the real-time clock module and the time matching operation also make the time correlation possible among multiple physiological signals without wire connection among multiple biosignal collecting units.

And, as a wireless communication is provided between the biosignal collecting units and the processing unit, the system according to the present invention becomes an on-line monitoring system, which is especially advantageous to EEG or PSG system. Besides, through connecting to network, the system even can achieve a remote on-line monitoring. Therefore, the medical personnel can instantly realize if the electrode/sensor installation is correct or the monitored patient's physiological condition.

In addition, since every biosignal collecting unit in the system is independent, the user can freely select the units to acquire the needed physiological signals without limitation, so that not only the scalability is significantly enhanced, the whole system also can be customized for saving cost.

The above examples and disclosure are intended to be illustrative and not exhaustive. These examples and description will suggest many variations and alternatives to one of ordinary skill in this art. All these alternatives and variations are intended to be included within the scope of the attached claims. Those familiar with the art may recognize other equivalents to the specific embodiments described herein which equivalents are also intended to be encompassed by the claims attached hereto.

What is claimed is:

1. A distributed multi-channel physiological monitoring and analyzing system, comprising:
an assembly of plural biosignal collecting units, for acquiring multiple physiological signals from an user, wherein each biosignal collecting unit comprises:
a processor;
at least a sensor/electrode for acquiring at least one channel of physiological signal;
a wireless module for data and commands communication;
a memory for storing the acquired physiological signals, and
a real-time clock module for providing a time base to sample the physiological signal and generating time axis information of the physiological signal; and
a processing unit, having a wireless module for receiving physiological signals from the biosignal collecting units for on-line monitoring,
wherein
a time matching operation is performed by aligning the time axis information among the processing unit and plural biosignal collecting units; and
one single set of time-correlated physiological data is formed based on the acquired multiple physiological signals and the result of the time matching operation for further analysis.

2. The system as claimed in claim 1, wherein the memory is a removable memory and the result of the time matching operation is stored therein, so that when the processing unit receives the physiological signals from the removable memories, the result of the time matching operation is also received thereby.

3. The system as claimed in claim 1, wherein the time matching operation is achieved by the processing unit wirelessly and simultaneously communicates with plural biosignal collecting units.

4. The system as claimed in claim 1, wherein the time matching operation zis achieved by the processing unit drives the processor of the biosignal collecting unit to produce a time stamp on the physiological signal in accordance with the time axis information provided by the processing unit, and after receiving plural physiological signals, the processing unit executes the time matching operation by aligning the time stamps.

5. The system as claimed in claim 1, wherein the time matching operation is achieved by the processing unit synchronizes the real time clock module with the time of the processing unit and simultaneously produces a time stamp on each physiological signal, and after receiving plural physiological signals, the processing unit executes the time matching operation by aligning the time stamps.

6. The system as claimed in claim 1, wherein the time matching operation is achieved by obtaining a time difference between each real time clock module and the time of the processing unit, and after receiving plural physiological signals, the processing unit executes the time matching operation through adjusting the time axes of the received multiple physiological signals according to the time differences thereof.

7. The system as claimed in claim 1, wherein the time matching operation is executed before, during and/or after physiological signal acquisition.

8. The system as claimed in claim 1, wherein the time matching operation is performed in the processing unit.

9. The system as claimed in claim 1, wherein the time matching operation is achieved by the processing unit communicates with each of the plural biosignal collecting units sequentially or simultaneously.

10. The system as claimed in claim 1, wherein the time matching operation is achieved by the processing unit communicates with one of the biosignal collecting units and the time-matched biosignal collecting unit communicates with other biosignal collecting units.

11. The system as claimed in claim 1, wherein the time matching operation is achieved by the processing unit communicates with one of the biosignal collecting units, and the time-matched biosignal collecting unit communicates with the next biosignal collecting unit until the time matching operations of all biosignal collecting units are completed.

12. The system as claimed in claim 1, wherein the time matching operation is achieved by the plural biosignal collecting units communicate with each other first, and then the processing unit communicates with plural biosignal collecting units.

13. The system as claimed in claim 1, wherein the wireless communication between the processing unit and the biosignal collecting units further includes an accuracy check of physiological signals and/or an impedance check.

14. The system as claimed in claim 13, wherein the processing unit is connected to a network for further connecting to a remote monitoring device so as to achieve a remote accuracy check of physiological signals and a remote impedance check.

15. The system as claimed in claim 1, wherein the processing unit is connected to a network for further connecting to a remote monitoring device so as to achieve a remote on-line physiological monitoring.

16. The system as claimed in claim 1, wherein the processing unit is adapted to provide an alarm function according to a monitored physiological condition.

17. The system as claimed in claim 16, wherein the processing unit is connected to a network for further achieving a remote alarm function.

18. The system as claimed in claim 1, further comprising an adapter device for receiving physiological signals and then transmitting the received physiological signals to the processing unit.

19. The system as claimed in claim 18, wherein the time matching operation is performed in the adapter device.

20. The system as claimed in claim 18, wherein the quantity of the adapter device is performed to be plurality, and plural physiological signals are divided into subgroups for transmitting to multiple adapter devices and then further transmitting to the processing unit.

21. The system as claimed in claim 1, wherein the biosignal collecting units communicate with each other in a wired or wireless manner, and the biosignal collecting units communicate with the processing unit in a wired or wireless manner.

22. The system as claimed in claim 1, further comprising an event button for marking an event generated during physiological signal acquisition.

23. The system as claimed in claim 22, wherein the event button is configured to integrate with one of the plural biosignal collecting units.

24. The system as claimed in claim 1, wherein the processing unit is configured to integrate with one of the plural biosignal collecting units.

25. The system as claimed in claim 1, wherein the processing unit is implemented to be a computer.

26. The system as claimed in claim 26, wherein the wireless module of the processing unit is built in or externally connected to the processing unit.

27. The system as claimed in claim 1, wherein the processing unit is composed of a computer and a embedded device which communicate with each other.

28. The system as claimed in claim 1, wherein the processing unit is implemented to be a standalone device.

29. A distributed multi-channel physiological diagnostic system, comprising:
an assembly of plural biosignal collecting units, for acquiring multiple physiological signals from an user, wherein each biosignal collecting unit comprises:
a processor;
at least a sensor/electrode for acquiring at least one channel of physiological signal;
a memory for storing the acquired physiological signals, and
a real-time clock module for providing a time base to sample the physiological signal and providing time axis information of the physiological signal; and
a processing unit, for receiving the physiological signals stored in the memories,
wherein
a time matching operation is performed by aligning the time axis information among the processing unit and plural biosignal collecting units; and
one single set of time-correlated physiological data is formed based on the acquired multiple physiological signals and the result of the time matching operation for further analysis.

30. The system as claimed in claim 29, wherein each biosignal collecting unit further comprises a wireless module for data and commands communication.

31. The system as claimed in claim 29, wherein the time matching operation is performed before biosignal acquisition.

32. The system as claimed in claim 29, wherein the time matching operation is performed after biosignal acquisition.
